(19) United States
(12) Patent Application Publication
(10) Pub. No.: US 2020/0179176 A1
(43) Pub. Date: Jun. 11, 2020

(54) KNITTED COMPRESSION ARTICLE

(71) Applicant: Julius Zorn Inc., Cuyahoga Falls, OH (US)

(72) Inventor: Siegfried LECHNER, Schrobenhausen (DE)

(73) Assignee: Julius Zorn Inc., Cuyahoga Falls, OH (US)

(21) Appl. No.: 16/703,065

(22) Filed: Dec. 4, 2019

(30) Foreign Application Priority Data
Dec. 5, 2018 (DE) 10 2018 130 932.5

(51) Int. Cl. A61F 13/06; A41D 19/00; A43B 7/00; A61F 13/10; D04B 1/28; D04B 1/18

(52) U.S. Cl. CPC A61F 13/064; A41D 19/0006; A43B 7/00; A61F 2013/00093; D04B 1/28; D04B 1/18; A41D 2500/10; A61F 13/104

(57) ABSTRACT

A knitted compression article with tubular or pocketlike receiving members for receiving at least one finger or toe of a wearer of the compression article, with a spacer element being disposed at least between two adjacent receiving members. Putting on such garment parts can be made easier and the fabrication of such garment parts can be simplified and, in particular, can be carried out in a single continuous knitting process without the need for subsequent sewing steps by providing that each receiving member has a front ply and a back ply and that the front ply and the back ply are seamlessly knitted to each other so as to create a tubular or pocketlike receiving member, with the spacer element or each spacer element being formed by connecting the front ply and the back ply to each other by a machine knitting technique.

### FIELD OF THE DISCLOSURE

The present disclosure relates to a knitted compression article that can be used, e.g., in scar therapy or in the treatment of burns or scalds or in reconstructive plastic surgery on a hand or foot, especially in postoperative compression therapy after reconstructive plastic surgery.

### BACKGROUND

In the treatment of scars caused, for example, by burns and scalds, it is customary to use compression articles, such as compression bandages, compression stockings or compression gloves, that exert a uniform pressure on the affected parts of the body and that can thereby inhibit the formation of proliferating scar tissue (keloids) or scar contractures. The compression articles used in the compression therapy of scars exert a therapeutically effective pressure on the scar, which is intended to prevent an unordered, excessive or reduced growth of body tissue that can lead to proliferating scars or scar contractures. In addition, compression therapy can also help to soften the scar tissue, it can fade reddish scar tissue, and it can reduce the thickness of the scar.

After scalding or burning of the hands and feet, scar tissue may proliferate in the space between adjacent fingers or toes, which can lead to the formation of so-called “webs.” To inhibit the formation of such proliferating scar tissue in the spaces between the fingers and the toes, compression therapy for scars on hands, for example, makes use of compression gloves that have so-called finger spacers. These finger spacers are disposed in the form of thick beadlike seams in between adjacent fingers of the glove. After fabrication of the compression glove, which can be knitted, for example, by means of circular knitting on a circular knitting machine, the seams, which form the finger spacers, are sewn between adjacent fingers of the glove to the outside of the glove. Thus, to form finger spacers, suitable seams of this type can be sewn, for example, between the 2^{nd} and 3^{rd} finger, the 3^{rd} and 4^{th} finger and/or the 4^{th} and 5^{th} finger. It is, however, also possible to provide finger spacers only for one or two spaces between the fingers. The spacer elements sewn between adjacent fingers of the compression glove, on the one hand, hold the fingers of a patient wearing the compression glove at a distance from each other and, on the other hand, exert pressure on the areas of the spaces between the fingers, which pressure is higher than the compression pressure exerted by the compression glove on the hand. The additional pressure exerted by the spacer elements on the area of the spaces between the fingers prevents, or at least suppresses, the formation of proliferating scar tissue and thus the formation of “webs” in the spaces between the fingers.

The fabrication of knitted compression gloves with spacer elements sewn into the areas of the spaces between adjacent fingers of the compression glove, however, is inconvenient and time-consuming since, after the glove has been knitted, the spacer elements have to be sewn on by hand or by machine in a separate production step. Furthermore, the spacer elements created by relatively thick seams reduce the wearing comfort.

Taking this as the starting point, the problem to be solved by the present disclosure is to make available a knitted compression article, for example, a compression glove or a sock liner for use in the compression therapy of hands or feet, that can be produced more easily and, in particular, in a single continuous knitting procedure without the need for subsequent sewing steps, and that comprises spacer elements, which exert an increased compression pressure on the areas of the spaces between adjacent fingers or toes of a patient so as to suppress keloids and, in particular, the formation of so-called “webs” and/or scar contractures, and which is more comfortable to wear.

### SUMMARY

Embodiments of the compression article as disclosed herein address these problems. Preferred embodiments of the compression article according to the present disclosure are disclosed herein. Potential applications and a method of its production are also disclosed.

The compression article according to the present disclosure is a knitted compression article, for example, in the form of a glove, a stocking or a sock liner, comprising a plurality of tubular or pocketlike receiving members for at least one finger or one toe of a patient wearing the compression article, with a spacer element being disposed at least between two adjacent receiving members. According to the present disclosure, each of the receiving members comprises a front ply and a back ply of a knitted material, with the front ply and the back ply being seamlessly knitted to each other so as to form the tubular or pocketlike receiving element and with the spacer element or each spacer element, which is disposed between adjacent receiving members, being formed during knitting by connecting the front ply and the back ply to each other by means of a machine knitting technique.

By connecting the front ply and the back ply of adjacent receiving members of the compression article according to the present disclosure to each other by means of a machine knitting technique, it is possible to incorporate the spacer elements while the compression article is being knitted. After completion of the knitting process, it is therefore no longer necessary to subsequently create the spacer elements by sewing seams into the spaces between adjacent receiving members on the outside of the compression article. The fabrication of the compression article is therefore simplified and can be implemented more rapidly and thus more cost-effectively. Furthermore, compression articles according to the present disclosure are more comfortable to wear because they have no subsequently attached seams; moreover, it has been shown that compression articles according to the present disclosure can be more easily and more quickly pulled over the hand or foot of a patient since the knitted-in spacer elements hold adjacent fingers or toes at a distance from each other while the compression article is being put on.

The spacer elements that are knitted into the compression article according to the present disclosure during the knitting process do not have any seams, which means that the compression article as a whole can be seamlessly fabricated. The seamless fabrication of the compression article offers improved wearing comfort and a more uniform distribution of the compression pressure exerted by the compression article on the hand or foot of the wearer.

A compression article comprising a front ply and a back ply and having a plurality of receiving members for the fingers or toes of a patient wearing the compression article can be seamlessly produced by means of knitting, for example, on a flat knitting machine with two needle beds disposed opposite to each other, i.e., a front needle bed and a back needle bed disposed opposite thereto.

When producing the compression article according to the present disclosure on a flat knitting machine comprising two oppositely disposed needle beds, a tubular main body with a front ply, which is knitted on the front needle bed of the flat-bed knitting machine, and a back ply, which is knitted on the back needle bed of the flat knitting machine, are formed to fabricate the compression article, with the front ply and the back ply of the tubular main body being seamlessly knitted to each other. Similarly, the receiving members for the fingers or the toes of a patient wearing the compression article are seamlessly knitted on a flat knitting machine, with the receiving members again having each a front ply and a back ply that are knitted on the front and the back needle bed of the flat knitting machine and that are seamlessly connected to each other so as to form the tubular or pocketlike receiving element. To create spacer elements between adjacent receiving members, the front ply and the back ply of the adjacent receiving members are connected to each other by means of a machine knitting technique in such a way that a flat spacer element is formed in the space between the adjacent receiving members. The flat spacer element between two adjacent receiving members may extend only over a single stitch or over a plurality of stitches of the knitted fabric of the knitted compression article. The front ply and the back ply of the two adjacent receiving members are preferably connected to each other by swapping the front ply and the back ply using a machine knitting technique.

Thus, the compression article according to the present disclosure comprises a knitted main body with tubular or pocketlike receiving members knitted thereto for receiving one or more fingers or one or more toes, with the main body of the compression article and the receiving members being knitted from the same basic knitted fabric with an inside face, which faces the body extremity (for example, the hand or foot of a wearer) when the compression article is worn, and an outside face disposed opposite thereto. The basic knitted fabric is favorably knitted along a stitch wale direction, which extends at least for the most part in the longitudinal direction of the receiving members, and has rows of stitches extending at right angles relative to the direction of the stitch wales. The basic knitted fabric is knitted from a stitch-forming knitting thread that may be elastic or inelastic. Preferably a compression-imparting elastic weft thread is incorporated or inserted into the basic knitted fabric. Each row of stitches of the basic knitted fabric preferably includes a weft thread. However, to produce, for example, a compression article with a lower level of compression pressure, it is also possible to incorporate a weft thread only into every second or third row of stitches, etc.

In the areas of the basic knitted fabric in which spacer elements are disposed, i.e., between two adjacent receiving members, the weft thread preferably is floating in the basic knitted fabric. As a result, the spacer elements disposed between the two adjacent receiving members maintain the fingers or toes of a wearer of the compression article at a distance from each other, which fingers or toes are accommodated in the receiving members, thereby suppressing the formation of proliferative scar tissue or so-called “webs.” In addition, it also makes it easier to put the compression article on a hand or foot by pushing the fingers or toes into the receiving members intended for this purpose since the spacer elements disposed between adjacent receiving members push the fingers or toes of the wearer slightly apart during insertion, thereby making it easier for the wearer of the compression article to slip his/her fingers or toes into the receiving members of the compression article provided for this purpose. Furthermore, putting on the compression article is made easier in that, if, during insertion, a pulling force is exerted on the toe part, the distribution of the pulling force is improved. Because of the connection by the spacer elements, the force exerted is also transmitted to the adjacent toe, whereby the distribution of the force is improved and putting on the compression article is made easier.

The basic knitted fabric of the compression article according to the present disclosure can favorably comprise a plurality of riblike elevations that, at least for the most part, extend parallel to and at a predefined distance from each other and along a longitudinal direction of the receiving members. The formation of such elevations, which favorably project from the outside of the basic knitted fabric, facilitates pulling the compression article over a hand or foot since the riblike elevations improve the longitudinal stretchability of the compression article.

To achieve the effect desired, the riblike elevations of the basic knitted fabric favorably extend over at least two stitches. The riblike elevations can be created, for example, by means of float stitches and/or tuck stitches of the knitting thread in the basic knitted fabric.

In a preferred embodiment of the present disclosure, the basic knitted fabric of the compression article is knitted in a knit/purl pattern (RL). However, to create the basic knitted fabric of the compression article according to the present disclosure, it is also possible to use any other conventional knitting constructions, for example, a 1:1 construction or a knit/knit construction (RR). Especially when using the preferred method of producing the compression article according to the present disclosure on a flat knitting machine, basically any knitting constructions that can be produced on a flat knitting machine can be used to create the basic knitted fabric and to optionally incorporate an elastic weft thread therein.

In addition to the preferred use in scar therapy or in the treatment of burns and scalds on hands and feet, the compression article according to the disclosure can also be used after reconstructive plastic surgery on a hand or foot and especially in postoperative compression therapy after reconstructive plastic surgery.

### BRIEF DESCRIPTION OF THE DRAWINGS

The features mentioned above as well as additional features, advantages and effects of the compression article according to the present disclosure follow from the practical examples described in greater detail below with reference to the appended drawings. The drawings show:

FIG. 1: A diagrammatic representation of a compression article according to the present disclosure in the form of a compression glove attached to the hand of a wearer;

FIG. 2: A diagrammatic representation of a compression article according to the present disclosure in the form of a toe part attached to the foot of a wearer;

FIGS. 3A to 3D: A number of different practical examples of knitting constructions of the basic knitted fabric of compression articles according to the present disclosure (FIGS. 3A to 3C) with an associated legend of the symbols used in the knit patterns of FIGS. 3A to 3C (FIG. 3D).

### DETAILED DESCRIPTION

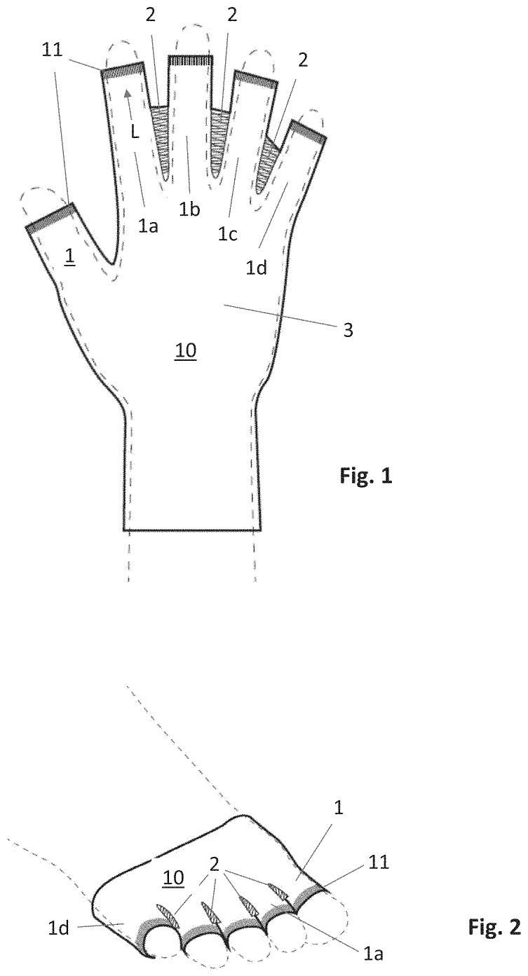

FIG. 1 shows a plan view of a first practical example of a knitted compression article according to the present disclosure in the form of a compression glove. The compression glove is attached to the hand of a wearer, which is indicated by the broken line. The compression glove comprises a tubular main body 10 that is adjoined by a total of five receiving members 1, 1a-1d. The receiving members 1, 1a-1d have each a tubular shape and serve to receive the five fingers of the hand of the wearer, with the first receiving member 1 serving to receive the thumb and the other four receiving members 1a-1d serving to receive the other four fingers of the hand. On their front (distal) ends, the receiving members 1 and 1a to 1d, which hereinafter will be jointly referred to by the reference character 1, are open so that, as shown in FIG. 1, the tips of the fingers project out of the open ends of the receiving members 1, 1a-1d. Knitted to each distal end of the receiving members 1 is a border in the form of a cuff 11. However, it is also possible to close the distal ends of the receiving members 1 with a dome-shaped cap so that the receiving members for the fingers of the hand are closed.

As FIG. 1 indicates, spacer elements 2 are disposed between the adjacent receiving members 1a and 1b, 1b and 1c, and 1c and 1d. The spacer elements 2 preferably extend over at least ⅓, most preferably over at least half, the length of the receiving members 1a to 1d (i.e., for example, over approximately half the dimension of the receiving members in their longitudinal direction L, as shown in FIG. 1). This allows the two adjacent receiving members 1a, 1b; 1b, 1c; 1c, 1d to be kept at a sufficiently large distance from each other by the spacer element 2 disposed in between.

The compression glove shown in FIG. 1 is seamlessly knitted in one piece, i.e., including the spacer elements 2, on a flat knitting machine comprising a front needle bed and a back needle bed disposed opposite thereto. The main body 10 and the adjoining receiving members 1 are each knitted from a basic knitted fabric 3 that comprises a front ply v and a back ply h that is seamlessly connected thereto. The front ply v of the basic knitted fabric 3 is knitted on the front needle bed of the flat knitting machine and the back ply h is knitted on the back needle bed. Seamlessly connecting the front ply v and the back ply h to each other by means of a machine knitting technique results in a tubular or pocketlike formation of the main body 10 and the receiving members 1, 1a-1d.

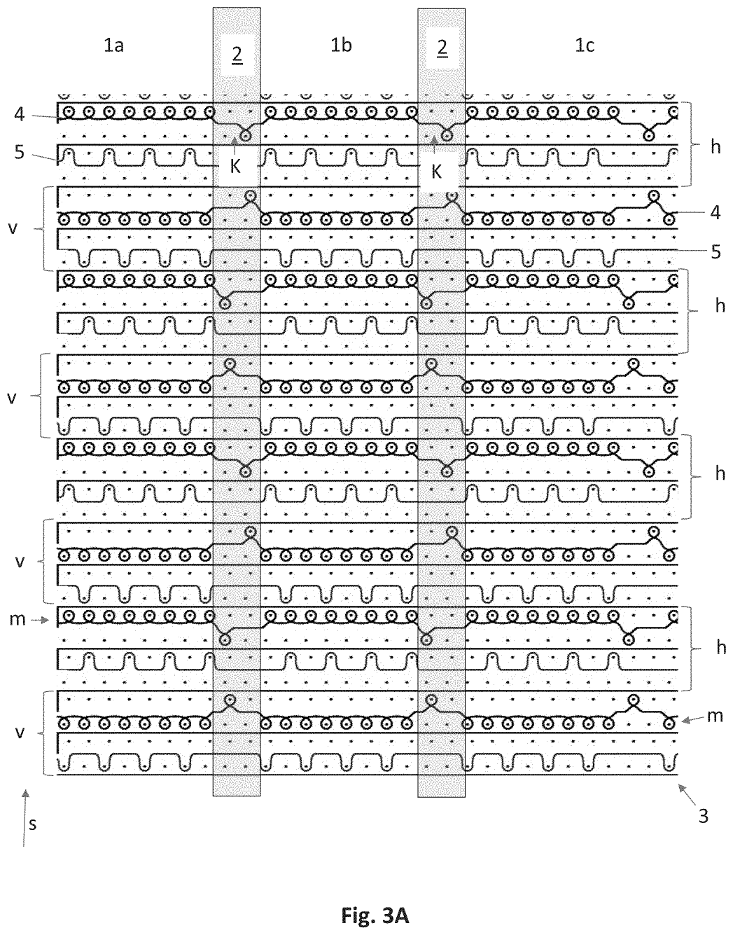

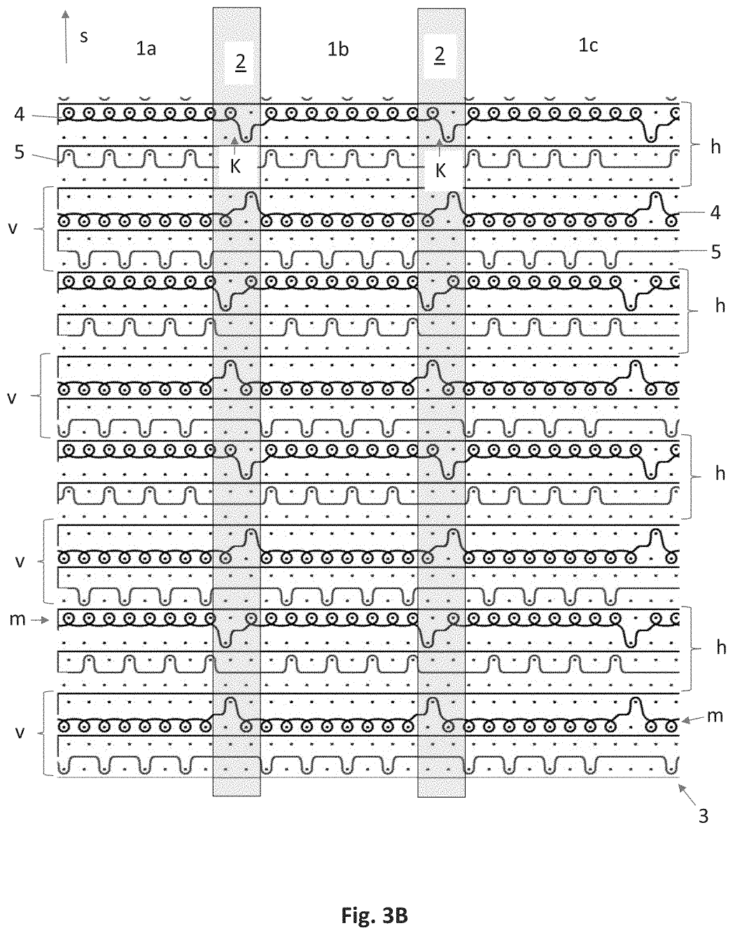

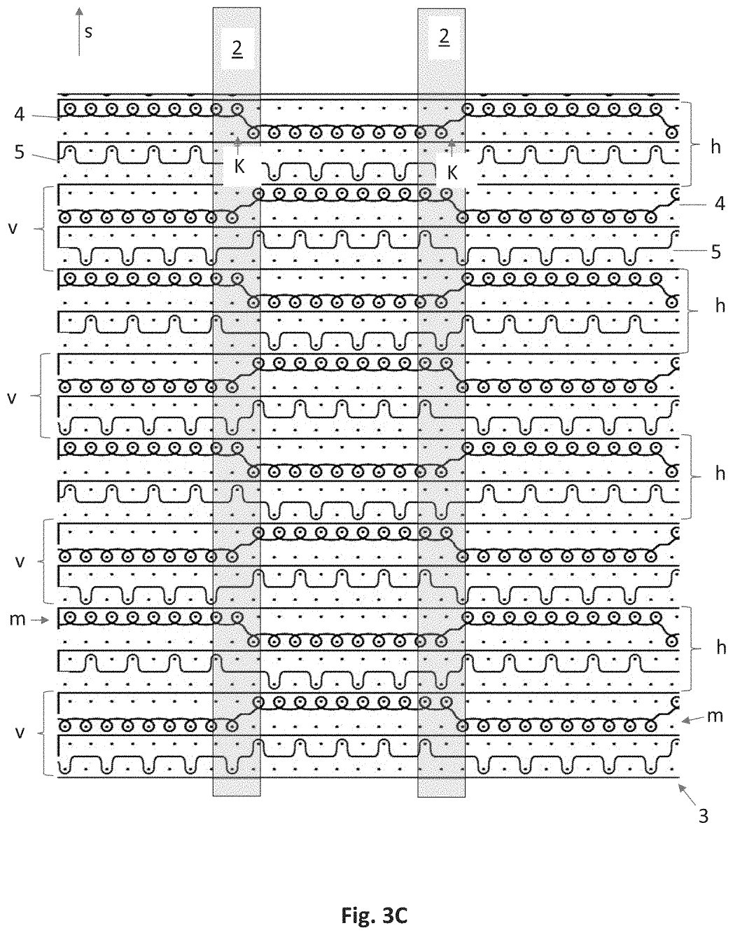

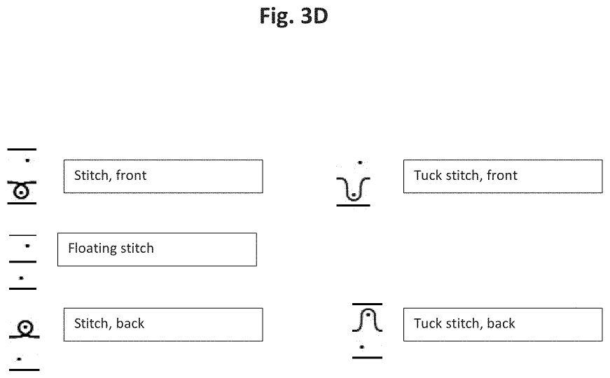

During the knitting of the compression article on a flat knitting machine, the spacer elements 2 formed in the spaces between the second and the third finger (1a, 1b), between the third and fourth finger (1b, 1c) and between the fourth and fifth finger (1c, 1d) are knitted into the basic knitted fabric 3, with each spacer element 2 being formed by connecting the front ply v and the back ply h of the adjacent receiving members 1a, 1b; 1b, 1c; 1c, 1d to each other by means of a machine knitting technique and, in particular, by swapping the front ply v and the back ply h. In the direction of the stitch rows m, the spacer elements 2 extend over at least two stitches (FIGS. 3A-3D).

To achieve a compressive action, an elastic weft thread is incorporated or inserted into the basic knitted fabric 3, from which the main body 10 and the receiving members 1 are knitted. The weft thread is incorporated or inserted into the basic knitted fabric 2 with an initial pre-tension. The weft thread used can be, for example, a covered yarn with a highly elastic core thread or a spandex or rubber thread, with the core thread and or the spandex or rubber thread preferably having a thickness in a range from 200 to 1500 dtex. The basic knitted fabric 3 is knitted from an elastic or an inelastic knitting thread. If an elastic knitting thread is used, it is possible to use, for example, a covered yarn with an elastic core thread. In this case, the weft thread is preferably thicker than the knitting thread.

To create the basic knitted fabric of the main body 10 and of the receiving members 1, any of the conventionally known knitting constructions can be used. The basic knitted fabric is preferably in a knit/purl pattern, and the weft thread is offset 1:1 and floating in the basic knitted fabric.

FIG. 2 shows a second practical example of a compression article according to the present disclosure in the form of a toe part with a tubular main body 10 and adjoining receiving members 1, 1a-1d. As in the practical example of FIG. 1, the tubular main body 10 and the five receiving members 1, 1a-1d are fabricated from a basic knitted fabric in a single knitting procedure, wherein knitting preferably takes place on a flat knitting machine with a front and a back needle bed, on which a front ply and a back ply of the main body 10 and of the receiving members 1, 1a-1d are knitted and seamlessly connected to each other so that tubular or pocketlike main bodies 10 and receiving members 1 are obtained. In the practical example of FIG. 2, the receiving members 1 are again open at their distal ends so that the toes of the wearer held in the receiving members 1, 1a-1d project out of the front end of the open receiving members 1.

FIGS. 3A-3D shows a number of different practical examples of knitting constructions which can be used to knit compression articles according to the present disclosure.

FIG. 3A shows a first practical example of a suitable knitting construction. In the area of the receiving members 1a-1c, the knitting construction shown consists of a basic knitted fabric 3, with spacer elements 2 extending between adjacent receiving members 1a, 1b; 1b, 1c. While the basic knitted fabric 3 is being knitted, the spacer elements 2 are formed by connecting a front ply v and a back ply h of the receiving members 1a, 1b and 1c to each other by means of a machine knitting technique.

The basic knitted fabric 3 shown in FIGS. 3A-3D can, for example, be seamlessly knitted on a flat knitting machine comprising a front and a back needle bed, with a front ply v of the receiving members 1a-1c being knitted on the front needle bed and a back ply h of the receiving members being knitted on the back needle bed. The front ply v and the back ply h of the receiving members 1a-1c are seamlessly connected to each other in such a fashion that a tubular or pocketlike receiving member is formed by the two plies v, h. As indicated in FIGS. 1 and 2, the distal ends of the receiving members 1a-1c can be open and be fitted with a cuff 11.

The knit pattern of FIG. 3A shows the formation of the spacer elements 2 by means of a machine knitting technique while the receiving members 1 are being knitted.

The basic knitted fabric 3 shown in FIG. 3A is knitted from an elastic or inelastic stitch-forming knitting thread 4. An elastic weft thread 5 extending in the direction of the stitch rows m of the basic knitted fabric 3 is incorporated into the basic knitted fabric 3. The basic knitted fabric 3 comprises a front ply v knitted on the front needle bed of the flat knitting machine and a ply h knitted on the back needle bed of the flat knitting machine. In the basic knitted fabric 3 shown in FIG. 3A, the weft thread is incorporated into each row of stitches m. It is, however, also possible to insert the weft thread 5 only into every second row of stitches or only into every third row of stitches, etc.

In the area of the tubular receiving members 1a, 1b, 1c, the knitting thread 4 forms stitches that, in the direction of the stitch rows m, are arranged side by side and, in the direction of the stitch wales s, are connected to each other to form loops. In the area of the receiving members 1a, 1b, 1c, the weft thread 5 alternately forms tuck stitches and float stitches.

In the area of the spacer elements 2, the front ply v and the back ply h of the receiving members 1a, 1b, 1c are swapped at crossover points K. At the crossover points K, the knitting thread 4, during knitting, is carried over from one ply (for example, the front ply v) to the ply disposed opposite thereto (back ply h), with the knitting thread 4 floating at the crossover point K and, after being carried over to the ply disposed opposite thereto, forming a stitch [at said crossover point]. In the stitch that, in the direction of the stitch row adjoins that stitch, the knitting thread 4 is carried back over to the original ply (front ply v) where it again forms a stitch. By carrying the knitting thread 4 over from one ply to the ply disposed opposite thereto, the tube of the tubular receiving member 1a, 1b, 1c is closed at the crossover points K, which, at the crossover points K, leads to the formation of a spacer element 2 that connects the adjacent receiving members 1a, 1b; 1b, 1c to each other. The spacer element 2 holds the adjacent receiving members at a distance from each other and causes the pressure in the area of the space between the adjacent fingers to increase when the compression glove is worn on the hand of a wearer.

FIG. 3B shows a second practical example of a knitting construction for a compression article according to the present disclosure in the area of adjacent receiving members 1a, 1b, 1c. As indicated in the practical example of FIG. 3A, the tubular or pocketlike receiving members 1a, 1b, 1c with the spacer elements 2 disposed in the intermediate spaces are formed from a basic knitted fabric 3, said basic knitted fabric 3 comprising a stitch-forming knitting thread 4 and an elastic weft thread 5 that is incorporated into each row of stitches m in the basic knitted fabric. The tubular or pocketlike receiving members 1a, 1b, 1c have a front ply v and a back ply h seamlessly adjacent thereto, with the front ply v being knitted on the front needle bed of a flat knitting machine and the back ply h being knitted on the back needle bed of the flat knitting machine. During knitting, the receiving members 1a, 1b; 1b, 1c disposed in the spaces between the spacer elements 2 are knitted in by swapping the front ply v and the back ply h of the receiving members 1a, 1b, 1c at the crossover points K. In contrast to the practical example of FIG. 3A, in this example, the stitch-forming knitting thread 4 is carried over, for example, from a back ply h at a crossover point K to the front ply v and forms a tuck stitch in the front ply v. In the stitch of the knitting thread 4 that, in the direction of the stitch row m, is disposed next to this tuck stitch, the knitting thread is again carried over to the back ply h where it forms a loop. It the area of the crossover point K, this leads to the formation of a spacer element 2 that, in the direction of the stitch row m, extends over two stitches.

In the direction of the stitch row m, the weft thread 5 alternately forms tuck and float stitches in the basic knitted fabric 3 and, in the area of the spacer elements 2 (i.e., at the crossover points K), it floats in the basic knitted fabric.

In the practical example of a basic knitted fabric 3 for a compression article according to the present disclosure shown in FIG. 3C, the oppositely disposed plies v and h are swapped in the areas of the crossover points K, at which points the stitch-forming knitting thread 4 is carried over from the front ply v to the back ply h (and vice versa). As shown in FIG. 3C, after the stitch-forming knitting thread 4 has been carried over, for example, from the back ply h to the front ply v at a crossover point K, this thread continues to be knitted as a stitch in the direction of the stitch row m on the front ply v, which causes the back ply h and the front ply v in adjacent receiving members 1a, 1b to be swapped. As in the practical examples of FIGS. 3A and 3C, the crossover of the stitch-forming knitting thread 4 at the crossover points K causes the tubular receiving member 1a, 1b, 1c to be closed and the spacer elements 2 to be formed between adjacent receiving members 1a, 1b; 1b, 1c.

It should be noted that the disclosure is not limited to the practical examples represented in the drawings and especially not to the preferred fabrication on a flat knitting machine with a front and a back needle bed. The basic knitted fabric of the compression article according to the present disclosure can, mutatis mutandis, also be fabricated as a circular knitted fabric on a circular knitting machine. Furthermore, to fabricate the basic knitted fabric 3, it is also possible to use different knitting constructions. The shape of the compression articles according to the present disclosure is not limited to the practical examples of a glove or a toe part shown in the drawings. For example, instead of a toe part, it is therefore also possible to fabricate a sock liner, a sock or a stocking with receiving members (that are open or closed at the distal end) for the toes of a wearer.

In addition, it is also possible for a compression article according to the present disclosure to be designed with receiving members for more than one finger or toe. Under certain circumstances, this can be useful if, for the purpose of stabilization, it were necessary to accommodate adjacent fingers or toes in a shared receiving member.

Compression articles according to the present disclosure can be used in scar therapy or for the treatment of burns or scalds or in reconstructive plastic surgery on a hand or a foot, especially in postoperative compression therapy after reconstructive plastic surgery, with the spacer elements of the compression article worn on a hand or foot exerting increased pressure on the areas of the spaces between the fingers or toes, thereby suppressing scar keloids and/or scar contractures.

1. A knitted compression article with a plurality of tubular or pocketlike receiving members for receiving at least one finger or toe of a wearer of the compression article, with a spacer element being disposed at least between two adjacent receiving members, wherein each receiving member comprises a front ply and a back ply, with the front ply and the back ply being seamlessly knitted to each other so as to form the tubular or pocketlike receiving member, and wherein each spacer element is formed by connecting the front ply and the back ply to each other by a machine knitting technique.

2. The compression article of claim 1, wherein the compression article as a whole is seamless and each spacer element is formed without seams.

3. The compression article of claim 2, wherein the compression article is knitted on a flat knitting machine having a front needle bed and a back needle bed disposed opposite thereto, with the front ply of the receiving members being knitted on the front needle bed and the back ply of the receiving members being knitted on the back needle bed.

4. The compression article of claim 1, wherein each spacer element is formed by swapping the front ply and the back ply.

5. The compression article of claim 1, wherein the compression article includes a basic knitted fabric having an inner face that, when the compression article is worn, faces a body extremity and an outer face disposed opposite thereto, with the basic knitted fabric being knitted along a stitch wale direction extending substantially in the longitudinal direction of the receiving members and having rows of stitches extending at right angles relative to the stitch wale direction.

6. The compression article of claim 5, wherein the basic knitted fabric is knit in a knit/purl pattern or in a knit/knit pattern and has a front ply and a back ply that is seamlessly connected to the front ply, with the front ply and the back ply being fabricated from at least one elastic or inelastic knitting thread.

7. The compression article of claim 5, wherein a weft thread is incorporated or inserted into the basic knitted fabric.

8. The compression article of claim 7, wherein the weft thread extends in each row of stitches or in every second row of stitches of the basic knitted fabric.

9. The compression article of claim 1, wherein in the direction of the stitch rows, each spacer element extends over at least two or three stitches.

10. The compression article of claim 1, wherein the front ply and the back ply of each receiving member has a knitting thread incorporated therein, which knitting thread, in the area of each spacer element, is carried over from one ply to the opposite ply at crossover points.

11. The compression article of claim 10, wherein in the area of each spacer element, the knitting thread is floating in a stitch adjacent to the crossover point in the row of stitches and/or at the crossover points, the knitting thread of a ply is cross-knitted in as a stitch or as a tuck stitch in the opposite ply.

12. The compression article as of claim 10, wherein in the area of each spacer element, the front ply and the back ply are swapped at the crossover points and vice versa.

13. The compression article of claim 1, wherein the compression article is a glove, with at least one of the plurality of receiving members for receiving a thumb and/or at least another of the plurality of receiving members for receiving a finger.

14. The compression article of claim 1, wherein the compression article is a stocking, sock, or sock liner, with at least one of the plurality of receiving members for receiving a toe and/or with at least another of the plurality of receiving members for receiving additional toes.

15. The compression article of claim 5, wherein each spacer element is knitted from the knitting thread of the basic knitted fabric by cross-knitting the knitting thread in from one ply to the opposite ply.

16. The compression article of claim 1, wherein each spacer element holds fingers or toes accommodated in the receiving members at a distance from each other and/or exert an increased pressure on the areas of the spaces between the fingers or toes.

17. The compression article of claim 1, wherein each receiving member extends over a predefined length in a longitudinal direction and wherein each spacer element extends over at least ⅓ the length of the respective adjacent receiving member.
